(19) United States
(12) Patent Application Publication
(10) Pub. No.: US 2015/0004066 A1
(43) Pub. Date: Jan. 1, 2015

(54) PERSONAL AIR FRESHENER

(71) Applicant: The Procter & Gamble Company, Cincinnati, OH (US)

(72) Inventors: Jessica Elizabeth LEON, Bellevue, KY (US); Stephan Gary BUSH, Liberty Township, OH (US); Dana Paul GRUENBACHER, Fairfield, OH (US); William Michael CANNON, West Harrison, OH (US); Andrew Isaac ROMLEIN, Cincinnati, OH (US)

(21) Appl. No.: 14/489,770

(22) Filed: Sep. 18, 2014

Related U.S. Application Data
(63) Continuation of application No. PCT/US2013/040899, filed on May 14, 2013.
(60) Provisional application No. 61/649,986, filed on May 22, 2012.

(51) Int. Cl. A61L 9/03

(52) U.S. Cl. CPC A61L 9/03
USPC 422/125; 512/4

(57) ABSTRACT

Personal scent delivery devices and perfume refills thereof allow a user to enjoy a personalized scent experience while not disturbing others that may be nearby (e.g., in an office setting).

### FIELD OF THE INVENTION

The present invention relates generally to personal air freshener dispensers and refills thereof.

### BACKGROUND OF THE INVENTION

Personal air freshener dispensers have been disclosed. However, there are several unmet needs for these devices that may include: maximizing the duration of the perfume refill (in view of relatively small dimensions of the device); providing the right balance of having a noticeable scent to the user while not offending people nearby; minimizing the effect of “scent habituation” that is typically associated with single scents; and a convenient way of storing refills.

### SUMMARY OF THE INVENTION

The present invention attempts to address these and other needs. In a first aspect of the invention, a blister pack comprising at least one perfume refill is provided. The pack has deformable shell that includes at least one blister associated with it. The perfume refill is contained in the blister. A rupturable material covers an opening of the blister. The rupturable material is configured to rupture upon dispensing of the perfume refill from the blister.

In a second aspect of the invention, a method of refilling a personal air freshening device with a perfume refill is provided. The steps include dispensing a personal air freshening device with a perfume refill. Another step includes inserting the dispensed perfume refill into the personal air freshening device, wherein the device is configured to receive the perfume refill.

In a third aspect of the invention, an article comprising a secondary package comprising an interior chamber, wherein the interior chamber contains a blister pack, wherein the blister pack contains a perfume refill in contained in at least one blister within the blister pack.

In a fourth aspect of the invention, a personal air freshening system is provided. The system comprises a perfume emitting device that houses a cavity configured to receive a perfume refill. Electric circuitry of the device includes a heater, and a power source interface capable of energizing the heater. A perfume refill receiver of the device is configured to receive a perfume refill. At least a portion of the receiver is disposed with the cavity of the housing. The dispenser is configured to dispense perfume from the perfume refill. The perfume refill is functionally attachable to the device. The perfume refill comprises a tray, wherein the tray comprises a cavity having a volume of about 0.1 cm³ to about 3 cm³. The cavity of the perfume refill contains perfume (and optionally a perfume carrier).

A fifth aspect of the invention provides for a perfume refill configured to be operatively and releasably connected to a personal air freshening device. The refill comprises a perfume tray having a cavity, wherein the cavity comprises a volume from about 0.1 cm³ to about 3 cm³. The cavity contains a perfume. The perfume comprises from about 50% to about 100% by weight of the perfume of at least one perfume ingredient, alternatively two, three, four, five, or more perfume ingredients, which has (have) a Kovat's Index from about 600 to about 1800.

A sixth aspect of the invention provides for a perfume refill configured to be operatively and releasably connected to a personal air freshening device. The refill comprises a perfume tray having a cavity, wherein the cavity comprises a volume from about 0.1 cm³ to about 3 cm³. The cavity contains a perfume. The perfume comprises perfume ingredients, and wherein the perfume ingredients are selected from: (i) a first group of ingredients having a boiling point of about 20° C. to about 250° C. and a ClogP value from about −2 to about 3; (ii) a second group of ingredients having a boiling point of about 20° C. to about 250° C. and a ClogP value from about 3 to about 9; (iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or (iv) a combination thereof.

A seventh aspect of the invention provides for a perfume refill configured to last about 40 hours, i.e., one work week.

### BRIEF DESCRIPTION OF THE DRAWINGS

FIG. 1 is a perfume refill.

FIG. 2 is a dispenser suitable for functionally receiving the perfume refill of FIG. 1.

FIG. 3 is the dispenser of FIG. 2 with a protective cover removed to expose for the USB port exposed.

### DETAILED DESCRIPTION OF THE INVENTION

Reference will now be made in detail to various embodiments of the present invention, examples of which are illustrated in the accompanying drawings wherein like numerals indicate the same elements throughout the views. All percentages, ratios and proportions herein are on a weight basis unless otherwise indicated.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

One aspect of the invention is to provide enough scent in the immediate space around the user to be detectable by the user but not so much as to be detectable by individuals around the user (such as in an office setting). Without wishing to be bound by theory, the desirability of the scent is very personal, that is, what is desirable to one person may not be desirable to another person. Having a personal zone of scent may provide the desired scent experience of the user without offending individuals around the user with an undesirable or unwelcome scent. Non-limiting examples of a personal zone of scent may be from about 10 m³ to about 0.1 m³, alternatively less than 9 m³, or 8 m³, or 7 m³, or 6 m³, or 5 m³, or 3 m³, or 2 m³, or 1 m³, alternatively from about 1 m³ to about 0.5 m³, alternatively combinations thereof.

Another aspect of the invention is to provide a single scent experience as to provide mitigation against olfactory saturation. The device of the present invention should emit scent with enough intensity and duration as to be detected by the user but not as to subject the individual to olfactory saturation (i.e., scent habituation).

Yet another aspect of the invention is to provide an individual a convenient refill pack as to allow the individual to conveniently select different scents yet keeping each individual refill from prematurely evaporating or commingling with other refills scents (thereby jeopardizing the fidelity of a specific refill's scent experience).

### Perfume

One aspect of the invention provides for a perfume or perfume composition (used herein interchangeably). Perfumes typically contain many perfume ingredients. As used herein the term “perfume” means any odoriferous material. In general, such materials are characterized by a vapor pressure less than atmospheric pressure at room temperatures. The perfumes employed herein will most often be liquid at room temperatures, but also can be solid such as the various camphoraceous perfumes known in the art. A wide variety of chemicals are known for perfumery uses, including materials such as aldehydes, ketones, esters, alcohols, terpenes and the like. Naturally occurring plant and animal oils and exudates comprising complex mixtures of various chemical components are known for use as perfumes, and such materials can be used herein. The perfumes herein can be relatively simple in their composition or can comprise highly sophisticated, complex mixtures of natural and synthetic chemical components, all chosen to provide any desired scent. Typical perfumes which can be used in the present invention comprise, for example, woody/earthy bases containing exotic materials such as sandalwood oil, civet, patchouli oil and the like. Other suitable perfumes are for example light, floral fragrances, e.g., rose extract, violet extract and the like. Perfumes can be formulated to provide desirable fruity odors, e.g., lime, lemon, orange and the like. In short, any chemically compatible material which emanates a pleasant or otherwise desirable scent can be used as a perfume in the present invention.

In one embodiment, the perfume comprises malodor elimination or malodor mitigation ingredients. Examples may include reactive aldehydes (e.g. US 2005/0124512), odor blocking materials, odor masking materials, or sensory modifying materials (e.g., ionones as disclosed in US 2005/0124512), and combinations thereof.

In one embodiment, the perfume can provide a short-term scent experience. A number of methods to control the intensity of scent within the present invention are envisioned. In some cases, this can be a result of the perfume composition, or perfume carrier (described further below), or a combination thereof. For example, the perfume can be formulated so that it has a more rapid release profile. Perfumes typically comprise one or more perfume ingredients. Often, these ingredients have different volatilities, boiling points, and odor detection thresholds. When a perfume composition volatilizes into the air, the ingredients with the higher volatilities (referred to as “top notes”) will be the ingredients that will volatilize and be detected by a person's sense of smell more quickly than the ingredients with lower volatilities (referred to as “middle notes”) and the ingredients with the lowest volatility (referred to as “bottom notes”). This will cause the character of the perfume to change over time since after the perfume is first emitted, the overall perfume character will contain fewer and fewer top notes and more bottom notes.

The perfume compositions can include ingredients that are suitably used in personal air freshening devices such as the present invention. The perfume ingredients are not limited but can be selected based on their Kovat's Index (“KI”) (as determined on 5% phenyl-methylpolysiloxane as non-polar silicone stationary phase). The KI places the volatility attributes of an analyte (e.g. component of a volatile composition) on a gas chromatography column in relation to the volatility characteristics of an n-alkane (normal alkane) series on that column. A typical gas chromatograph (“GC”) column is a DB-5 column available from Agilent Technologies of Palo Alto, Calif. By this definition, the KI of a normal alkane is set to 100n, where n is the number of carbon atoms in the n-alkane. The KI of an analyte, x, eluting at time t′, between two n-alkanes with number of carbon atoms “n” and “N” having corrected retention times t′ₙ and t^{t}_{N} respectively, will then be calculated as:

$$KI = 100 ( n + \frac{log {t}_{x}^{′} - {log}_{n}^{′}}{log {t}_{N}^{′} - log {t}_{n}^{′}} )$$

On a non-polar to slightly polar GC stationary phase, KI of analytes are correlated with their relative volatility. For example, analytes with smaller KIs tend to be more volatile than those with larger KIs. Ranking analytes with their corresponding KI values gives a good comparison of analyte evaporation rates in liquid-gas partitioning systems. The volatile composition according to the present invention can have at least one ingredient with a KI value of about 600 to about 1800, or about 800 to about 1700, or about 900 to about 1600. The volatile composition can comprise about 50% to about 100%, or about 70% to about 100%, or about 80% to about 100% of one or more ingredients having these KI values.

Rather than, or in addition to Kovat's Index, the perfume ingredients can be selected based on their boiling point (or “B.P.”) and their octanol/water partition coefficient (or “P”). The boiling point referred to herein is measured under normal standard pressure of 760 mm Hg. The boiling points of many perfume ingredients, at standard 760 mm Hg can be found in “Perfume and Flavor Chemicals (Aroma Chemicals),” written and published by Steffen Arctander, 1969. In one embodiment, the perfume comprises from about 50% to about 100% by weight of at least one perfume ingredient, alternatively two, three, four, five, or more perfume ingredients, which has (have) a Kovat's Index from about 600 to about 1800.

The octanol/water partition coefficient of a perfume ingredient is the ratio between its equilibrium concentrations in octanol and in water. The partition coefficients of the perfume ingredients used in the volatile composition may be more conveniently given in the form of their logarithm to the base 10, logP. For purposes of the present invention, the calculated logP (“ClogP”) is determined by using the Biobyte CLOG P program contained in Daylight software version 4.94 or latest version currently available for license from Daylight Chemical Information Systems (“Daylight”) or any later version of the Biobyte CLOG P program that is available for licensing from anyone, including Daylight. For purposes of determining the ClogP values, the measurement shall not include those ingredients in the perfume that do not provide a pleasant smell, such as, by way of example, solvents, diluents, antioxidants, preservatives, and surfactants. Non-limiting examples of these materials are ethyl alcohol, carbitol, diethylene glycol, dipropylene glycol, diethyl phthalate, triethyl citrate, isopropyl myristate, and benzyl benzoate.

The perfume composition might comprise perfume ingredients selected from one or more groups of ingredients. A first group of ingredients may comprise perfume ingredients that have a boiling point of about 20° C. to about 250° C., or a boiling point of about 25° C. to about 240° C., or a boiling point of about 30° C. to about 235° C. The first group of ingredients may comprise perfume ingredients that have a ClogP value from about −2 to about 3 or from about −1 to about 2.5. In certain embodiments, perfume ingredients selected from the first group of perfume ingredients when present may be present at a level of from about 20% to about 100% by weight of the perfume composition, or from about 40% to about 100% by weight of the perfume composition, or from about 50% to about 100% by weight of the perfume composition.

A second group of ingredients might comprise perfume ingredients that have a boiling point of about 20° C. to about 250° C., or a boiling point of about 25° C. to about 240° C., or a boiling point of about 30° C. to about 235° C. The second group of ingredients may comprise perfume ingredients that have a ClogP value from about 3 to about 9 or from about more or about 3.5 to about 7. In certain embodiments, the second perfume ingredient when present may be present at a level of from about 20% to about 100% by weight of the perfume composition, or from about 40% to about 100% by weight of the perfume composition, or from about 50% to about 100% by weight of the perfume composition.

A third group of ingredients might comprise perfume ingredients that have a boiling point of about 250° C. to about 400° C., or about 260° C. to abut 375° C. The third group of ingredients may comprise perfume ingredients that have a ClogP value of about from about −2 to about 3 or from about −1 to about 2.5. In certain embodiments, perfume ingredients selected from the third group of perfume ingredients when present may be present at a level of about 0.5% to about 90% by weight of the perfume composition or about 1% to about 80% by weight of the perfume composition.

A fourth group of ingredients might comprise perfume ingredients that have a boiling point of about 250° C. to about 400° C., or about 260° C. to about 375° C. The fourth group of ingredients may comprise perfume ingredients that have a ClogP value from about 3 to about 9 or from about more or about 3.5 to about 7. In certain embodiments, perfume ingredients selected from the fourth group of perfume ingredients when present may be present at a level of about 0.5% to about 90% by weight of the perfume composition or about 1% to about 80% by weight of the perfume composition.

In one embodiment, the perfume comprises perfume ingredients, wherein the perfume ingredients are selected from: (i) a first group of ingredients having a boiling point of about 20° C. to about 250° C. and a ClogP value from about −2 to about 3; (ii) a second group of ingredients having a boiling point of about 20° C. to about 250° C. and a ClogP value from about 3 to about 9; (iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or (iv) a combination thereof.

Another means of controlling the release of perfume is by encapsulating the perfume in the form of capsules non-limiting examples of which include microcapsules, or starch encapsulates. There are a number of means in which the capsules can be designed to release the scent. For example the scent can be released by either rupturing the capsules or by diffusion through the capsule wall. In another example the scent is released from the microcapsule when moisture in the air causes the capsule wall to rupture. Alternatively, the capsules can be ruptured by peeling off an adhesive layer that causes the microcapsule to tear or rupture. The capsule size and material properties can also be adjusted to control the diffusion. Suppliers of microcapsules may include International Flavors & Fragrances (IFF), Reed Pacific, and Appleton.

In one embodiment, the perfume further comprises a malodor elimination chemical compound. See e.g., U.S. Patent Publications claims priority to and/or published from the following U.S. patent application Ser. Nos. 12/962,691; 13/081,559; 12/904,261; 13/006,644; 13/249,616; 61/622,030.

### Perfume Carrier

One aspect of the invention provides using a perfume carrier to carry the perfume composition. The perfume and perfume carrier can be contained in a cavity portion of a tray. In one embodiment, the perfume carrier can be an absorbent pad or gauge or paper. In other embodiment, the material used as the perfume carrier is a polymeric composition.

One example of a polymeric composition is one comprising a copolymer obtained from at least one monomer comprising an ether group and at least one monomer not comprising an ether group. Optionally a compatible plasticizer may be used. The term “monomer comprising an ether group” includes all those monomers wherein at least an oxygen atom is bonded to two different carbon atoms none of which is connected to another oxygen atom, including those where the oxygen atom is included in a cyclic epoxide structure such as for example ethylene oxide, propylene oxide, tetramethylene oxide and the like. The term “monomer comprising an ether group” also includes those molecules, oligomers or polymers which contain an ether group inside the molecule and might be further polymerized.

These copolymers can be both block and non-block copolymers, also copolymers formed by the grafting of polyether groups onto polymer structures, copolymers where the polyether group is comprised in a side chain or in a crosslink and copolymers where ether monomers are randomly copolymerized with non-ether monomers are suitable copolymers for the present invention. Among preferred copolymers obtained from at least one monomer comprising an ether group and at least one monomer not comprising an ether group which are suitable for the present invention are, for example, polyether amide copolymers, polyether ester copolymers, polyether urethanes, sulfonated polyether ester copolymers, polyether ester amide copolymers, copolymers formed by the polymerization of polyether acrylates with other acrylic monomers/oligomers, etc. Wherein a polyether group is present, preferred polyether groups are those with monomers which contain more than 2 carbon atoms, more preferred are those with monomers which contain more than 3 carbon atoms. Most preferred polyether containing groups are polypropylene glycol and, even more preferred, polytetramethylene glycol.

The monomer comprising an ether group in the copolymers suitable for the present invention represents at least 5% of the total weight of the copolymer, preferably at least 10% more preferably at least 15%.

Preferred copolymers for the present invention are poly-ether-amide block copolymers (e.g. Pebax®, especially Pebax® 2533 SA 1), poly-ether-ester-amide block copolymers, polyether polyester block copolymers (e.g. Hytrel®) polyurethane copolymers containing polyether blocks (e.g. Estane®) or mixtures thereof. Among the various copolymers of this type are preferred those which have a preferred polyether group as mentioned above. Therefore most preferred copolymers are those in which the polyether group is a polypropylene glycol or a polytetramethylene glycol.

Preferably the polymeric composition of the present invention comprises from 5% to 75%, more preferably from 10% to 50% by weight of the polymeric composition, of the copolymer obtained from at least one monomer comprising an ether group and at least one monomer not comprising an ether group; from 5% to 60%, preferably from 10% to 40% by weight of the polymeric composition, of the suitable plasticizer or blend of plasticizers, and more than 10%, preferably more than 20%, more preferably more than 30% of a volatile material; the perfume is preferably comprised up to a maximum percentage of 90% by weight of the polymeric composition.

The polymeric compositions of the present invention preferably are thermoplastic polymeric compositions. These can be manufactured by using any known process for manufacturing thermoplastic polymeric compositions and will typically comprise the steps of melting the polymer and then blending the plasticizer and the perfume to form a homogeneous mass that is then cooled to obtain the polymeric composition according to the present invention. Among thermoplastic compositions preferred are those which have low melt temperature and viscosity and therefore are processable as hot melts. In these systems the loss of volatile perfume ingredients upon blending is minimized.

Optional components include other copolymers that can be included in the formulations to improve their properties for example to increase adhesion or compatibility with substrates. To this purpose preferred optional copolymers are those featuring both polar and non-polar groups, for example: copolymers of ethylene and at least one other vinyl or acrylic monomer, copolymers of styrene and at least one other vinyl or acrylic monomer, copolymers of poly(vinyl alcohol), polyamides, copolymers of poly(vinyl pyrolidone), polyacrylates, copolymers of polyvinyl ethers), ionomers, polyester amide copolymers, etc. Further optional ingredients such as other polymers or copolymers, fillers, crosslinkers, pigments, dyes, antioxidants and other stabilizers, etc can also be added to provide desired properties to the composition.

In one embodiment, the perfume carrier may comprise a plasticizer. Suitable plasticizers for use in the present invention include citric acid esters, low molecular weight polyesters, polyethers, liquid rosin esters, aromatic sulfonamides, phthalates, benzoates, sucrose esters, derivatives of polyfunctional alcohols (where polyfunctional means having two or more hydroxyl groups), adipates, tartrates, sebacates, esters of phosphoric acid, fatty acids and diacids, fatty alcohols and diols, epoxidised vegetable oils etc, and mixtures thereof. Without wishing to be bound by theory, the different polarity of the different compatible plasticizers (measurable with any method known to those skilled in the art, for example water/octanol partition coefficient) can be used to tune the polarity of the polymeric matrix in order to provide a better match with the polarity of the perfume.

### Perfume Tray

One aspect of the invention provides from a perfume refill, wherein the refill comprises a tray, wherein the tray comprises a cavity, wherein the cavity contains a perfume (and optionally a perfume carrier). The tray may also have a grip tab (graspable by the user's fingers) to facilitate the insertion and removal of the refill from a perfume dispensing device. In one aspect of the invention, the tray has at least one cavity suitable for holding a perfume/perfume carrier. In one embodiment, two, three or more cavities are provided to hold multiple perfumes. In one embodiment, the cavity comprises a volume from about 0.5 cm³ to about 2 cm³, alternatively from about 0.7 cm³ to about 1 cm³, alternatively from about 0.4 cm³ to about 3 cm³, alternatively from about 0.1 cm³ to about 3 cm³, alternatively combinations thereof.

In one embodiment, the volume of the perfume/perfume carrier contained in the cavity of the refill is from about 0.3 cm³ to about 0.9 cm³, alternatively from about 0.4 cm³ to about 0.8 cm³, alternatively from about 0.5 cm³ to about 0.7 cm³, alternatively combinations thereof. In another embodiment, the weight of the perfume/perfume carrier contained in the cavity of the refill is from about 0.3 g to about 0.9 g, alternatively from about 0.4 g to about 0.8 g, alternatively from about 0.5 g to about 0.7 g, alternatively combinations thereof.

The surface that is exposed of the perfume/perfume carrier contained in the cavity is important, in some embodiments, in delivering the desired amount of scent over the desired length of time. In one embodiment, the exposed surface area of a perfume/perfume carrier contained in the cavity of the refill is from about 100 mm² to about 330 mm², alternatively from about 130 mm² to about 300 mm², alternatively from about 150 mm² to about 250 mm², alternatively from about 175 mm² to about 225 mm², alternatively from about 200 mm² to about 260 mm², alternatively from about 210 mm² to about 240 mm², alternatively from about 180 mm² to about 220 mm², alternatively about 232 mm², alternatively about 196 mm², alternatively combinations thereof.

The tray can be formed from any formable plastic, paper, metal, film, or other material, laminate, or combination thereof, capable of maintaining its structural integrity and not adversely reacting when subjected to perfume carrier and/or perfume materials. In one embodiment, the tray may be formed from a thermoplastic or thermoset plastic. A thermoplastic tray can be manufactured by a conventional plastic invention molding or thermoforming processes, for example.

In one embodiment, the perfume tray may be formed, preferably thermoformed, by materials that generally are not permeable to perfumes or volatile compositions. In another embodiment, the tray is covered by a protective layer of a material that is generally not permeable to perfume or volatile compositions. Example may include a metal foil (e.g., aluminum foil).

In one embodiment, the perfume tray is thermoformed. In another embodiment, the perfume tray has a cavity, preferably centrally located, and a surrounding peripheral flange. The perfume carrier can be first placed in the cavity of the tray, and a permeable cover can then be positioned over the perfume tray (or at least the opening of the cavity portion of the tray) to enclose the perfume carrier contained in the cavity to form a perfume tray enclosing the perfume carrier. In another embodiment, the perfume tray does not have a cover, or a releasable attached non-permeable cover over at least the opening of the cavity portion of the perfume tray, or a releasable attached non-permeable cover over the permeable cover. Examples of permeable materials suitable as a permeable cover may include high, medium, and low density, polyethylene ultra low density polyethylene and ethylene vinyl acetate copolymer. Other examples include microporous polytetraflouralethylene (“PTFE”), and other microporous membranes as known in the art. The degree of permeability of a particular polymer material can be increased by adding impurities such as calcium carbonate into the polymer material before the permeable cover is formed in order to open up additional spaces between the polymer chains thereby increasing the permeability of the material. Additional permeability may also be a by-product of coloring the layer by introducing solid micro particles of pigment, which also tends to open up spaces between the polymer chains. See e.g., U.S. Pat. No. 5,804,264. The permeable layer may be attached to the tray by heat seal, alternatively heat sealed to the surrounding peripheral flange of the tray.

In one embodiment, there is an additional release layer releasably attached to the permeable cover or the tray directly when a permeable cover is not present. The release layer can be easily pulled away from the permeable membrane/tray by the user. The release layer is a material that is generally non-permeable to perfume at ambient temperature. It may be releasable attached by an adhesive or a weak heat bond to the permeable cover. It may have a pull-tab to facilitate removal.

In one embodiment, the refill is made of the following layer of thermoform materials: APET-LDPE-EVOH-LDPE. A perfume impermeable layer is preferably between the LDPE and EVOH. Zobele Group may be a manufacturer.

### Blister Pack

As used herein, the term “blister pack” is to be understood to refer to a package in which discrete quantities or units of the perfume refill are stored in a “blister” and dispensed by applying force to the blister to expel or dispense the perfume refill from the blister pack. It is contemplated that the present invention can be utilized with commonly available blister packaging technology, and can be adapted for use with known blister pack configurations in addition to use with blister packs specifically designed to be incorporated into the present configuration. Without wishing to be bound by theory, the blister pack is a convenient way to keep the perfume from becoming prematurely volatized and yet a convenient way to dispose perfume refill. In one embodiment, only the perfume and perfume carrier are contained within the blister of the blister pack (and not the tray). Alternatively, the tray containing the perfume (and optionally the perfume carrier, plasticizer, etc) is contained in the blister of the blister pack.

In one embodiment, the blister pack includes a deformable shell including at least one pocket or blister associated therewith. A unit of perfume refill can be stored in the at least one blister. A rupturable material can be coupled over an opening of the blister. The rupturable material can be configured to rupture upon dispensing of the unit of perfume refill from the blister. The unit of perfume refill can be dispensed from the blister by the application of force to a top portion of the blister. As force is applied to the blister, the blister collapses inward, and the unit of perfume refill is forced into the rupturable material. The rupturable material is ruptured as a result, and the unit of perfume refill is free to be withdrawn from the blister.

The blister pack container can be formed in a variety of manners known to those having ordinary skill in the art and can be formed from a variety of suitable materials. In general, the blister pack can include a shell material that is formed to include a series of indentations or pockets (e.g., individual “blisters”) that are each configured to receive one or more units of perfume refill. The shell material can be, but is not necessarily, formed of a plastic material such as a polyvinyl chloride laminate. A generally thin metal foil, or other rupturable material, can be attached or adhered to the shell material around and across openings in the depressions or indentations (e.g., “blisters”). While not so required, a series of perforations can be formed in the blister pack container to allow each blister to be individually separated into a discrete unit. A substantially continuous sheet of rupturable material can be applied over the entire undersurface of the blister pack, and each blister can be separated from the remainder of blisters with the portion of the rupturable material covering the particular blister remaining intact and uncompromised. See e.g., See U.S. Pat. No. 7,802,682 B2.

### Package for Securing Blister Pack

One aspect of the invention comprises a secondary package for receiving and containing one or more of the blister packs (containing perfume refills) described herein. Such a package can have branding and other necessary information for the product to be sold. It also can some level of protection for the blister pack to be pre-maturely ruptured during shipping and handling. It can also serve as a convenient way to pack a plurality of blister packs (including “club” sizes or as a “variety” pack of different scents).

In one embodiment, the package comprises an interior chamber adopted to receive and contain a blister pack within the interior chamber. The package may have a thumb/finger indent to allow the user more easily grasp the blister pack contained within the package. There may also be a retaining system between the package and the blister pack to help retain the blister pack within the secondary package. For example, there may be a button on the blister pack and an aperture on the blister pack (or vice versa) adopted to engage and cooperate to resist disengagement between the blister pack and the secondary package when the blister pack is contained in the chamber of the secondary package.

The secondary package may be made from cardboard, plastic, or combinations thereof.

### Personal Air Freshener Dispenser

One aspect of the invention provides a dispenser configured to receive a refill and to dispense scent from the perfume contained in the cavity of the perfume refill, wherein the refill is configured to be operatively received in the dispenser. The dispenser is configured to as to operatively receive the perfume refill, alternatively configured to receive a plurality of refills, alternatively configured to receive a single refill, wherein the refill has a two least two cavities, with the first cavity contains a first perfume and the second cavity contains a second perfume. The first perfume and second perfume provide different scents.

The dispenser may comprise a heating element capable of applying heat to the perfume contained in the perfume refill. The heating element is configured to be in thermal communication with the refill when the refill is functionally received within the dispenser. Alternatively, the dispenser may comprise a spray system including a piezoelectric transducer. The dispenser may operative in a passive mode and/or active mode (i.e., heating or spraying the perfume).

The dispenser may comprise a power supply and a control circuit for controlling the heating element and/or piezoelectric transducer.

The dispenser may comprises a connector capable of connecting to a base station, wherein the base station may include a computer, smart phone, personal digital assistant, and the like. One example of a connector is a USB key or another peripheral that is capable of benefiting from the power supply of the computer, for example.

Light or other additional indicia can be a way to allow the user the dispenser is properly attached to the power source.

The dispenser may have a power or control setting. There may be a timer (for e.g., to have the device turn on only during office hours), scent intensity control (e.g., controlling vents or power or timing of the passive/active modes).

Non-limiting examples of a dispenser may include: US 2010/0176213 A1; US 2010/0019057 A1

In one embodiment, a personal air freshener dispenser is provided. The dispenser may comprise housing having a cavity (configured to receive a perfume refill), electrical circuitry disposed within the cavity, wherein electric circuitry includes a heater, and a power source interface capable of energizing the heater, a perfume refill tray, wherein at least a portion of the tray is disposed with the cavity of the housing, and wherein the dispenser is adapted to dispense perfume from the perfume tray. In another embodiment the power source interface is a USB interface that is in turn coupled to a power source to energize the heater (in the active mode). In another embodiment, the housing comprises a lower housing and an upper housing, wherein the upper housing is secured to the lower housing to define the cavity therebetween.

Optionally the dispenser may have computer memory and may contain computer readable files pre-loaded onto the memory.

In one embodiment, the refill provides up to one week, preferably two weeks, preferably 30 days, alternatively more than 30 days of longevity, alternatively from about 1 week to less than about 70 days. In another embodiment, the refill provides from about 30 hours to about 40 hours, alternatively from about 35 hours to about 45 hours, alternatively about 40 hours, alternative up to about 40 hours, alternatively combinations thereof. Without wishing to be bound by theory, internal unpublished consumer research suggests that consumer do not care to replace refill more frequently than once per week with many consumers working about 40 hours per week. In yet another embodiment, the article comprises a timer that automatically turns off the article after 8 hours (after initially being turned on by the user). Alternatively, the timer turns the article on or off at certain predetermined times (i.e., “on” at 9 am and “off” at 5 pm). These times by controlled by the user and/or pre-set at the factory.

In one embodiment, the dispenser comprises vents or other orifices that provide fluid communication between the perfume/perfume carrier contained in the refill and that of the external airspace outside of the dispenser. Without wishing to be bound by theory, it may be important, in some embodiments, the relationship between the planar surface area of the vents and other such openings in the dispenser (collectively “air vents”) and that of the perfume/carrier contained in the cavity of the refill when the refill is operably attached to the dispenser. The term “air vent” also include the relevant area of the refill receiving channel of the dispenser. In non-limiting examples, there is generally a 1 mm to 3 mm vertical gap when the refill is functionally attached to the dispenser (relative t the article when it is operatively attached to a USB port of a e.g., desktop computer). The planar surface of these air vents is the smallest planar surface area that can be measured for the respective air vent. The planar surface area of all the air vents of the article is used to calculate the total planar surface area. Generally, the greater the total planar surface area of the collective air vents of the article, the greater the rate and degree of diffusion of the perfume (since there is less physical restriction of the perfume to diffuse from the refill). The converse is also generally true. In one embodiment, the total surface area of the air vents is from about 50 mm² to about 210 mm², alternatively from about 70 mm² to about 190 mm², alternatively from about 90 mm² to about 170 mm², alternatively from about 110 mm² to about 150 mm², alternatively 120 mm² to about 140 mm², alternatively from about 100 mm² to about 150 mm², alternatively about 130 mm², alternatively combinations thereof. Based on the surface area discloses, ratio ranges of the total planar surface area of the air vents: surface area of the exposed perfume/perfume refill contained in the cavity of the refill, wherein the refill is operably received in the dispenser, are also contemplated within the scope of the present invention.

### Mitigating Scent Habituation

A problem with many freshening devices is that a user may become desensitized to a scent after being continuously exposed to the scent over a period of time. The consumer no longer notices the scent. One way of mitigating this problem is by alternating scents. This way, once the consumer becomes desensitized to a first scent, a second scent is introduced and the first scent is turned off. After awhile, the second scent is turned off, and the first scent is again turned on. This process is repeated. Third or fourth or more different scents can also be used. The scent emissions between the two or more scents can be overlapping or non-overlapping. In one embodiment, the refill of the present invention contains two or move cavities with each cavity containing a different perfume composition. The device alternates heating between the cavities to control scent emission. See e.g., U.S. Pat. Nos. 7,223,361; 8,016,207; 7,981,367; 8,061,628; 8,119,064.

Another aspect of the invention provides for pulsating a single scent or perfume. Pulsating can be accomplished, in on embodiment, by turning on and off the heater/heating element that heats the perfume contained the cavity of the refill. In one embodiment, the heater is turned on from about 2 minutes to about 12 minutes, alternatively from about 4 minutes to about 11 minutes, alternatively from about 5 minutes to about 10 minutes, alternatively from about 6 minutes to 9 minutes, alternatively from 7 minutes to 10 minutes, alternatively from 5 minutes to 9 minutes, alternatively combinations thereof. In another embodiment, the heater is turned off from about 4 minutes to about 12 minutes, alternatively turned off from about 5 minutes to about 10 minutes, alternatively from about 5 minutes to about 12 minutes, alternatively from about 6 minutes to 9 minutes, alternatively from 7 minutes to 10 minutes, alternatively from 5 minutes to 9 minutes, alternatively combinations thereof. In yet another embodiment, the time ratio from the heater on: heater off is from about 1:1 to about 1:20, alternatively form about 1:1 to about 1:10, alternatively from about 1:1 to about 1:5, respectively, alternatively combinations thereof.

In some embodiments, the temperature is an important factor for solving the aforementioned problems. The temperature, measured at the heating element and when the heating element is on, is from about 60° C. to about 70° C., alternatively from about 62° C. to about 68° C., alternatively from about 64° C. to about 66° C., alternatively about 65° C., alternatively combinations thereof. Without wishing to be bound by theory, temperatures within the aforementioned ranges provide the amount of heat necessary to volatize perfume to be noticed by the user but not so much as to go beyond the desired “personal space” the invention is generally designed to deliver.

### EXAMPLE 1

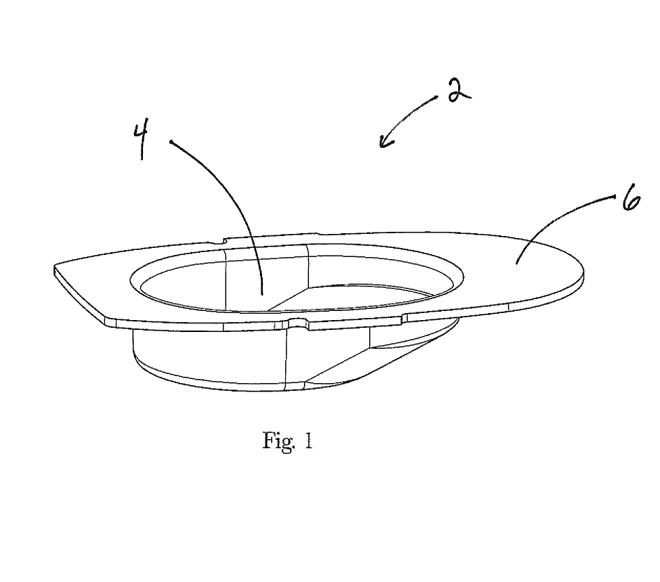

FIG. 1 is an example of a perfume refill (2). The perfume refill (2) has a cavity (4) suitable for containing a perfume/perfume carrier.

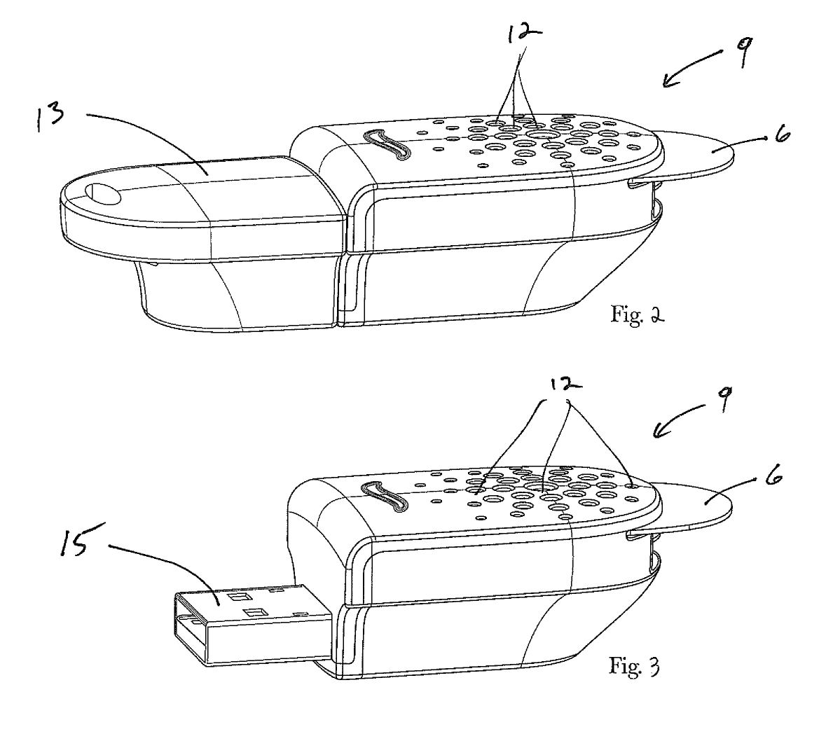

FIGS. 2 and 3 are an example of a perfume emitting device (9). The perfume refill (2) is functionally received in the device (9) with only a gripable tab (6) exposed. A plurality of air vents (12) allow the perfume to diffuse from the interior of the device (9) from the refill (2) contained therein. FIG. 2 has a USB interface (13) functionally attached to the device (9) whereas FIG. 3 has the cover removed to expose the USB interface (15).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A blister pack containing a perfume refill comprising:
a deformable shell including at least one blister associated therewith;
the perfume refill, contained in the at least one blister;
a rupturable material covering an opening of the blister, the rupturable material being configured to rupture upon dispensing of the perfume refill from the blister.

2. The blisterpack of claim 1, further comprising a backing layer, disposed on an opposite side of the rupturable material from the blister, the backing layer being configured to protect the rupturable material from rupturing prior to removal of the backing layer.

3. The blisterpack of claim 1, further comprising a plurality of blisters, each having the perfume refill contained therein.

4. The blisterpack of claim 3, further comprising a substantially continuous sheet of rupturable material coupled over an opening of each of the plurality of blisters.

5. The blisterpack of claim 3, wherein a first blister of the plurality of blisters contains a first perfume refill having a first perfume and a second blister of the plurality of blisters contains a second perfume refill having a second perfume.

6. The blisterpack of claim 3, wherein one of the blisters is configured to be separated from adjacent blisters while maintaining the rupturable material over the opening of the one blister in an un-ruptured condition.

7. The blisterpack of claim 3, wherein the perfume refill comprises a cavity, wherein the cavity contains perfume.

8. The blisterpack of claim 7, wherein the cavity further contains a perfume carrier.

9. A personal air freshening system comprising:
(a) a perfume emitting device comprising a housing, wherein the housing houses a heating element, a power source interface, and a perfume refill receiver, wherein the power source interface is configured to energize the heating element, wherein the heating element is configured to be in thermal communication with a perfume refill when the refill is functionally and releasably received in the perfume refill receiver of the device; and
(b) the perfume refill is configured to be functionally and releasably receivable in the perfume emitting device, wherein the perfume refill comprises a cavity, wherein the cavity comprises a volume from about having a volume of about 0.1 cm³ to about 3 cm³, and wherein the cavity contains perfume.

10. The system of claim 9, wherein the tray is a thermoform tray, and the power source interface is a USB interface; and wherein the perfume refill comprises a gripping tab that is not housed by the housing of the device when the refill is functionally received in the device.

11. A perfume refill configured to be functionally and releasably receivable in a personal air freshening device, wherein the refill comprises a tray having a cavity, wherein the cavity comprises a volume from about 0.1 cm³ to about 3 cm³, wherein the cavity contains a perfume, wherein the perfume comprises from about 50% to about 100% by weight of the perfume of at least one perfume ingredient having a Kovat's Index from about 600 to about 1800.

12. The refill of claim 11, wherein the perfume comprises at least three perfume ingredients having a Kovat's Index from about 600 to about 1800.

13. The refill of claim 12, wherein the perfume comprises at least five perfume ingredients having a Kovat's Index from about 600 to about 1800.

14. A perfume refill configured to be operatively and releasably connected to a personal air freshening device, wherein the refill comprises a perfume tray having a cavity, wherein the cavity comprises a volume from about 0.1 cm³ to about 3 cm³, wherein the cavity contains a perfume, wherein the perfume comprises perfume ingredients, and wherein the perfume ingredients are selected from:
(i) a first group of ingredients having a boiling point of about 20° C. to about 250° C. and a ClogP value from about −2 to about 3;
(ii) a second group of ingredients having a boiling point of about 20° C. to about 250° C. and a ClogP value from about 3 to about 9;
(iii) a third group of ingredients having a boiling point of about 250° C. to about 400° C. and a ClogP value from about −2 to about 3; a fourth group of ingredients having a boiling point of about 250° C. to about 400° C. and a ClogP of about 3 to about 9; or
(iv) a combination thereof.

15. The perfume refill of claim 13, wherein the cavity further contains a copolymer comprising at least one ether-substituted monomer and at least one non-ether substituted monomer.

16. The perfume refill of claim 15, wherein the copolymer is selected from the group consisting of: poly-ether-amide block copolymers; poly-ether-ester-amide block copolymers; polyether polyester block copolymers; polyurethane copolymers containing polyether blocks; or mixtures thereof.

17. The perfume refill of claim 16, wherein the copolymer comprises poly-ether-amide block copolymers.

18. The perfume refill of claim 17, wherein the cavity further contains a copolymer comprising at least one ether-substituted monomer and at least one non-ether substituted monomer.

19. The perfume refill of claim 18, wherein the copolymer is selected from the group consisting of: poly-ether-amide block copolymers; poly-ether-ester-amide block copolymers; polyether polyester block copolymers; polyurethane copolymers containing polyether blocks; or mixtures thereof.

20. The perfume refill of claim 19, wherein the copolymer comprises poly-ether-amide block copolymers.
